(19) United States
(12) Patent Application Publication
(10) Pub. No.: US 2003/0103931 A1
(43) Pub. Date: Jun. 5, 2003

(54) Oral agent for adsorbing phosphorus and food comprising the same

(75) Inventors: Shinichi Takasaki, Yokohama-shi (JP); Masami Todokoro, Minamata-shi (JP); Takuji Uesako, Itano-gun (JP); Ippei Yamaoka, Tokushima-shi (JP); Masaru Kobayashi, Naruto-shi (JP)

Correspondence Address: SUGHRUE MION, PLLC, 2100 Pennsylvania Avenue, NW, Washington, DC, 20037-3213, US

(73) Assignee: CHISSO CORPORATION

(21) Appl. No.: 10/200,547

(22) Filed: Jul. 23, 2002

(30) Foreign Application Priority Data
Jul. 24, 2001 (JP) P. 2001-223302

(51) Int. Cl. A61K 31/785

(52) U.S. Cl. 424/78.3

(57) ABSTRACT

An oral agent for adsorbing phosphorus, which comprises a polylysine as an active ingredient; a food which comprises a polylysine; and a method for reducing a phosphorus concentration in blood and a method for excreting phosphorus, using the oral agent for adsorbing phosphorus.

### FIELD OF THE INVENTION

The present invention relates to an oral agent for adsorbing phosphorus. More particularly, it relates to an oral agent for adsorbing phosphorus having an excellent activity to adsorb over-ingested phosphorus in the digestive tract and thereby accelerating excretion of the phosphorus into feces, and to its application to food.

### BACKGROUND OF THE INVENTION

A case in which the serum phosphorus concentration becomes 5.0 mg/dl is called hyperphosphatenia, and its cause is classified into (i) increase in the phosphorus release from cells, (ii) increase in the phosphorus load from outside the body and (iii) decrease in the phosphorus excretion from the kidney.

Phosphorus as a main anion in the cells is released into blood caused by cell disruption (e.g., hemolysis after chemotherapy of malignant tumors, striated muscle necrosis) and becomes a cause of hyperphosphatenia.

Also, the conditions at the time of excess phosphorus load from the outside of the body are the same. Since hypocalcemia is also generated when hyperphosphatenia is present, increased phosphorus ingestion is not desirable, but it is the present situation that dairy products such as milk and fish paste products such as fish meat known as phosphorus-rich food must be ingested in a large amount from the viewpoint of calcium reinforcement because of their high calcium content. In addition, vitamin D toxication can also generate hypercalcemia and hyperphosphatenia by increasing absorption of calcium and phosphorus from intestines.

The hyperphosphatenia secondarily causes hypocalcemia, induces secondary hyperparathyroidism and becomes a factor to induce osteoporosis by drawing calcium from bones.

Also, when renal function disorders such as renal insufficiency and the like are present, the serum phosphorus concentration increases when, e.g., GFR (glomerular filtration rate) becomes 30% or less of the normal level. What is more, since production of vitamin D is hindered in renal insufficiency, hypocalcemia is generated and secretion of PTH (parathyroid hormone) is increased secondarily, but the excretion activity of PTH is gradually attenuated and excess PTH alone becomes conspicuous. Also, in the case of hypoparathyroidism and pseudohypoparathyroidism, they have no PTH activity so that they become a cause of hyperphosphatenia.

Since the presence of excess phosphorus inside the body exerts bad influences on the human body in this manner, great concern has been directed toward the development of a phosphorus adsorbing agent which can excrete the excessively presenting phosphorus into the outside of the body by effectively inhibiting absorption of phosphorus into the body. As examples of the phosphorus adsorbing agent, aluminum preparations such as dry aluminum hydroxide gel and the like, calcium preparations such as calcium carbonate and the like, and magnesium preparations such as magnesium hydroxide and the like, are conventionally known, but these preparations have problems such as high side effects and low phosphorus adsorbing ability.

### SUMMARY OF THE INVENTION

An object of the present invention is to provide a novel agent for adsorbing phosphorus and food, which is safe without side effects and has high phosphorus adsorbing ability.

As a result of extensive studies, the present inventors have found that a polylysine, particularly a polylysine crosslinked by a crosslinking agent, has excellent phosphorus adsorbing ability, is safe and can be used in food and medicaments, thereby resulting in the accomplishment of the present invention.

Accordingly, the present invention relates to the following (1) to (20).

(1) An oral agent for adsorbing phosphorus, which comprises a polylysine as an active ingredient.

(2) The oral agent for adsorbing phosphorus according to (1), wherein the polylysine is crosslinked by a crosslinking agent.

(3) The oral agent for adsorbing phosphorus according to (2), wherein the polylysine is crosslinked by a crosslinking agent and has a granular shape.

(4) The oral agent for adsorbing phosphorus according to (2), wherein the polylysine is crosslinked by a crosslinking agent and has a plate shape.

(5) The oral agent for adsorbing phosphorus according to (2), wherein the crosslinking agent is an epoxy compound.

(6) The oral agent for adsorbing phosphorus according to (5), wherein the crosslinking agent is epichlorohydrin.

(7) The oral agent for adsorbing phosphorus according to (5), wherein the crosslinking agent is ethylene glycol diglycidyl ether.

(8) The oral agent for adsorbing phosphorus according to (1), wherein the polylysine is poly(α-lysine).

(9) The oral agent for adsorbing phosphorus according to (1), wherein the polylysine is poly(ε-lysine).

(10) A food which comprises the polylysine according to (1).

(11) The food according to (10), wherein the polylysine is crosslinked by a crosslinking agent.

(12) The food according to (11), wherein the polylysine is crosslinked by a crosslinking agent and has a granular shape.

(13) The food according to (11), wherein the polylysine is crosslinked by a crosslinking agent and has a plate shape.

(14) The food according to (11), wherein the crosslinking agent is an epoxy compound.

(15) The food according to (14), wherein the crosslinking agent is epichlorohydrin.

(16) The food according to (14), wherein the crosslinking agent is ethylene glycol diglycidyl ether.

(17) The food according to (10), wherein the polylysine is poly(α-lysine).

(18) The food according to (10), wherein the polylysine is poly(ε-lysine).

(19) A method for reducing a phosphorus concentration in blood, which comprises administering an effective amount of the oral agent for adsorbing phosphorus of (1) to human or an animal.

(20) A method for excreting phosphorus, which comprises adsorbing phosphorus in the digestive tract by the oral agent for adsorbing phosphorus of (1) and allowing the phosphorus to be excreted together with the adsorbing agent.

### DETAILED DESCRIPTION OF THE INVENTION

The polylysine to be used in the present invention may be either α type or ε type shown below. It is preferably ε type, because ε type is cationic (pKa 7.6) compared to α type.

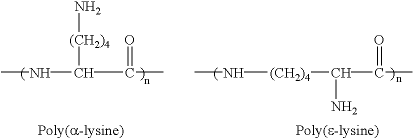

Also, it may be a microbial origin produced by a bacterium belonging to the genus Streptomyces or a product obtained by chemical synthesis, but a microbial origin produced by a bacterium belonging to the genus Streptomyces is particularly preferable, because it is ε type, has high biocompatibility due to its biodegradability and can be obtained in a large amount with low cost. Specific examples of the production method of polylysine include the methods described in JP-A-53-72896, JP-A-63-49097, JP-A-3-143398, JP-A-6-86686, JP-A-8-163992, JP-A-9-154593, JP-A-10-174596, JP-A-10-210995, JP-A-11-137287 and the like.

Although the molecular weight of the polylysine used in the present invention is not particularly limited, it is preferably from 500 to 1,000,000, particularly preferably from 1,000 to 10,000. According to the present invention, the polylysine may be made into a crosslinked structure, or two or more species of the polylysine may be mixed at an optional mixing ratio. Particularly, a polylysine crosslinked with a crosslinking agent is preferable in terms that it is easy to take due to improved bitterness and it is safe with no side effects due to less degradation in the digestive tract.

Any crosslinking agent may be used in preparing the polylysine crosslinked with a crosslinking agent (a polylysine having a crosslinked structure; hereinafter referred to as “crosslinked polylysine”), so long as it can form the structure. The crosslinking agent to be used in the present invention is not particularly limited, and its examples include bifunctional reagents hexamethylene diisocyanate and epoxy compounds such as ethylene glycol diglycidyl ether, epichlorohydrin and the like. Among these, epoxy compounds such as ethylene glycol diglycidyl ether, epichlorohydrin and the like are particularly preferable crosslinking agents, because they are easily ring-opened and inexpensive.

The reaction method and condition using a crosslinking agent can be optionally selected in response to the used crosslinking agent, but a polylysine can be made into a crosslinked structure generally by stirring the materials in an inert dispersion medium or by carrying out suspension polymerization of the polylysine with the crosslinking agent in an aqueous solution. The amount of the crosslinking agent to be added in that case is not particularly limited, too, but is preferably from 2 to 100% by mol, particularly preferably from 10 to 30% by mol.

Specifically, a crosslinked polylysine can be produced by adding a crosslinking agent to an aqueous polylysine solution. The polylysine is water-soluble in nature and becomes insoluble by forming a crosslinked structure when a crosslinking agent is added. The used polylysine may be either α type or ε type as described above and its molecular weight is not particularly limited, too. Also, the polylysine concentration in the aqueous solution is not particularly limited, but it is preferably from 10 to 70% by weight. Since the reaction temperature and reaction time vary depending on the reactivity of used crosslinking agent and its adding amount, they are not particularly limited, but when epichlorohydrin is used as the crosslinking agent for example, a crosslinked polylysine suitable for the present invention can be obtained by stirring the materials at a reaction temperature of around 40° C. for about 16 hours.

According to the phosphorus adsorbing agent of the present invention, the effect of the present invention can be obtained, so long as it contains the polylysine or crosslinked polylysine regardless of its shape. The shape may be a plate shape such as a board or a film, a granular shape or a porous block shape. When the polylysine or crosslinked polylysine of the present invention is applied to a drug or food, a granular shape or a plate shape is preferable. The particle diameter in this case is not particularly limited, but it is generally from 0.1 to 1,000 μm, preferably from 5 to 500 μm.

The thickness when made into a plate shape is not particularly limited, but in general, it is preferably from 100 to 1,000 μm.

Preferred physical properties of the crosslinked polylysine are as follows.

(1) Ion exchange capacity: 4 to 5 meq/g

(2) Degree of swelling: 1 to 20 ml/g

(3) Nitrogen content: 10 to 20%

As described above, the polylysine or crosslinked polylysine is safe showing no side effects and, at the same time, shows excellent phosphorus adsorbing ability, particularly an activity to effectively adsorb phosphorus which is present in the digestive tract and to accelerate its excretion when it is orally used.

Accordingly, the polylysine or crosslinked polylysine is effective as an oral agent for adsorbing phosphorus and can be contained in various articles such as food. Particularly, it can alleviate phosphorus ingestion by patients of various diseases caused by over-ingestion of phosphorus, such as hyperphosphatenia, renal insufficiency, osteoporosis and the like, and is useful as a medicament for use in their prevention and treatment. Also, since the polylysine or crosslinked polylysine excretes phosphorus in a large amount in feces, it is particularly effective for the excretion of phosphorus in patients such as renal diseases having reduced renal function and the like.

The oral agent for adsorbing phosphorus of the present invention can be produced by processing the polylysine or crosslinked polylysine obtained by the method described in the above into various forms by a usual method. For example, it may be a solid product, a liquid product, an emulsified product, a paste product or a jelly product.

Also, in addition to medicaments, the oral agent for adsorbing phosphorus of the present invention can be effectively applied to food. All of those which can be taken immediately as such, which are taken after cooking and premixed materials for food production use are included in the phosphorus adsorbing agent of the present invention. Among foods containing the oral agent for adsorbing phosphorus of the present invention, the solid food may be in any one of powdery shape, granular shape and solid shape, and its examples include various confectionery such as biscuits, cookies, cakes, snacks, rice crackers and the like, bread and powdered drinks (e.g., powdered coffee and cocoa). Also, examples of the liquid product, emulsified product, paste product and jelly product include various drinks such as juices, carbonated drinks, lactic acid drinks and the like. Paste products and jelly products are preferred.

When the oral agent for adsorbing phosphorus of the present invention is used particularly as a medicament, it can also be made into general pharmaceutical preparations such as tablets, powders, granules, fine particles, solutions and the like. The pharmaceutical preparations can be produced by making the polylysine or crosslinked polylysine to be used in the present invention into desired preparations in accordance with a usual method, together with a pharmaceutically acceptable carrier.

The polylysine or crosslinked polylysine to be used in the present invention has an ability to excrete and absorb about 50 mg of phosphorus by about 1 g. Accordingly, using this as a measure, it is preferable to take a dose of approximately 1 to 20 g, particularly about 2 to 10 g, based on the polylysine or crosslinked polylysine.

The polylysine or the polylysine crosslinked by a crosslinking agent effectively inhibits absorption of phosphorus in the digestive tract to thereby reduce the absorption ratio inside the body, and particularly has excellent activity to excrete the phosphorus into feces, so that an oral agent for adsorbing phosphorus, which is safe with no side effects and also has excellent phosphorus adsorbing ability, can be produced by containing this in an effective amount.

Also, when the polylysine or the polylysine crosslinked by a crosslinking agent is contained in an effective amount, it becomes a preventive or therapeutic agent for diseases caused by over-ingestion of phosphorus. Particularly, the phosphorus adsorbing agent of the present invention shows a large phosphorus excreting amount into feces, so that it is markedly effective in patients having reduced phosphorus excretion from the kidney such as renal insufficiency and the like.

In order to describe the present invention further in detail, the phosphorus adsorbing agent of the present invention containing the polylysine or crosslinked polylysine and production methods of medicaments and food containing the same are cited in the following as examples, although the present invention is not limited thereto.

### EXAMPLE 1

Phosphorus adsorbing effects of polylysine and crosslinked polylysine were comparatively examined using normal rats.

(1) Synthesis of Crosslinked Polylysine

(1-1) Synthesis of Crosslinked Polylysine A

To 60 ml of a 25% by weight aqueous solution of ε-polylysine (manufactured by Chisso Corporation), 10 g of epichlorohydrin and 5 ml of a 50% by weight aqueous sodium hydroxide solution were added, followed by stirring at 40° C. for 16 hours to obtain a water-insoluble mass polymer. The thus obtained mass polymer was collected by filtration, washed with ether and then dried. The dried mass polymer was pulverized into an appropriate size and then screened to obtain 30 g of particles having a size of 100 to 200 μm, which are called spherical ε-polylysine A.

(1-2) Synthesis of Crosslinked Polylysine B

In 100 ml of heptane, 0.5 g of sorbitan monooleate was dissolved. To the solution, 20 ml of a 25% by weight aqueous solution of ε-polylysine (manufactured by Chisso Corporation), followed by stirring at a temperature of 30 to 35° C. for dispersion. To the mixture, 1 ml of ethylene glycol diglycidyl ether was added and allowed to react at 40 to 50° C. for 1 hour. The thus obtained reaction product was washed by filtration (twice with methanol, thrice with hot water, twice with pure water), and then the thus obtained crosslinked polylysine was dried overnight in an oven of 80° C. to obtain 4.5 g (yield 95%) of dried crosslinked polylysine, which is called granular ε-polylysine B.

(1-3) Synthesis of Crosslinked Polylysine C

To 6 ml of a 25% aqueous solution of ε-polylysine, 0.6 ml of ethylene glycol diglycidyl ether was added, followed quickly mixing, and the resulting mixture was poured into a dish of 9 cm in diameter and then allowed to stand overnight at 40° C. The thus obtained membrane was washed with methanol and water to obtain a crosslinked polylysine having a thickness of about 1 mm, which is called plate shape ε-polylysine C.

(2) Materials and Methods

As the experimental animals, 8-week-old Wistar male rats (Charles River Japan, Inc.) were used. The animals were fastened overnight and then transferred into separate metabolic cages and subjected to 3 days of preliminary breeding under restricted feeding (20 g/rat/day). Thereafter, the animals were checked for their body weights and divided into groups (n=6/group) using their feeding amounts and body weights during the acclimation period as indexes. That is, they were divided into three groups of a control group, a polylysine group and a crosslinked polylysine groups.

Rats of each group were reared for 4 days under restricted feeding with respective test feed (20 g/rat/day). That is, the control group was fed with a commercial powder feed (CRF1 Powder, manufactured by Oriental Yeast Co., Ltd.) supplemented with 2% by weight of sodium dihydrogenphosphate, and each test feed group with a mixed feed prepared by adding a test substance (a polylysine 100% powder (the ε-PL-1 described in JP-A-10-306160, page 4, right side column, lines 16 to 32) or the crosslinked polylysine B) to a final concentration of 3% by weight to the commercial powder feed product supplemented with 2% by weight of sodium dihydrogenphosphate.

The amount of the feed ingested was recorded every day, and collection of feces and urine was carried out on the 3rd and 4th days after commencement of the test.

(3) Results

The evaluated items were balance value of phosphorus (Pi) and amount of feed ingested.

Amount of ingested phosphorus, amount of excreted phosphorus in urine and amount of excreted phosphorus in feces in each group on the 4th day are shown in Table 1. Also, comparison of dairy amounts of feed ingested (g/time) in the polylysine group and crosslinked polylysine group is shown in Table 2.

TABLE 1

| | | | Crosslinked |
|---|---|---|---|
| | Control group | Polylysine | polylysine |
| Items evaluated | (n = 6) | group (n = 6) | group (n = 6) |
| Ingested | 241.0 ± 1.3 | 236.9 ± 0.9 | 238.2 ± 0.4 |
| phosphorus | | | |
| (mg/day) | | | |
| Excreted | 73.4 ± 8.4 | 64.8 ± 5.6 | 56.5 ± 4.0 |
| phosphorus in | | | |
| urine (mg/day) | | | |
| Excreted | 133.4 ± 10.5 | 162.8 ± 9.7 | 158.2 ± 14.7 |
| phosphorus in | | | |
| feces (mg/day) | | | |

TABLE 2

| | First day | Second day | Third day | Fourth day |
|---|---|---|---|---|
| Polylysine group | 16 to 17 g | 14 to 17 g | 20 g | 20 g |
| Crosslinked | 20 g | 20 g | 20 g | 20 g |
| polylysine group | | | | |

In comparison with the control group, decrease in the excretion of phosphorus in urine was found in each of the polylysine group and crosslinked polylysine group. On the other hand, regarding the amount of phosphorus excreted in feces, increase in the excretion of phosphorus in feces was found in each of the polylysine group and crosslinked polylysine group in comparison with the control group. Accordingly, these compounds are effective particularly as phosphorus excreting agents for patients of renal insufficiency.

Also, it can be seen from Table 2 that the amount of feed ingested in the polylysine group shifted at a low level until on the 2nd day after commencement of the test, in comparison with the crosslinked polylysine group. That is, improvement in the amount of feed ingested can be obtained by making polylysine into a crosslinked structure by a crosslinking agent.

(4) Summary

It was shown from these results that the polylysine and crosslinked polylysine have an activity to inhibit absorption of phosphorus inside the body by excreting the ingested phosphorus from feces.

### EXAMPLE 2

Production of Fine Subtilaes:

The term “part(s)” as used in the following examples means part(s) by weight.

A mixture of 70 parts of the polylysine powder or granular crosslinked polylysine used in Example 1, 20 parts of lactose and 10 parts of corn starch was subjected to fluidized bed granulating in 5% by weight aqueous solution of hydroxypropylmethylcellulose to obtain respective fine subtilaes.

### EXAMPLE 3

Production of Biscuits:

A loaf of dough was prepared by mixing 8 parts of shortening with 18 parts of sugar, adding thereto 42 parts of soft wheat flour, 7.5 parts of the polylysine powder or granular crosslinked polylysine used in Example 1, 0.8 part of baking powder, 16 parts of eggs, 1 part of glucose and 25 parts of water, followed by stirring. The dough was rolled to a thickness of 5 mm, cut with a die to a size of 16 to 17 g per piece and then baked for 32 to 36 minutes in an oven of 90° C. As a result, biscuits of about 12 g per piece were obtained. It is calculated that a biscuit of 12 g contains 1 g of the polylysine or the granular crosslinked polylysine. That is, about 50 mg of phosphorus can be adsorbed by eating one piece of the biscuits.

### EXAMPLE 4

Production of a Drink:

A drink containing the polylysine or crosslinked polylysine was obtained by dissolving 12.5 g of granulated sugar, 0.2 g of citric acid crystals and 1 g of the polylysine powder or granular crosslinked polylysine used in Example 1 in 100 ml of ion exchange water, and then bottling the solution and sterilizing it at 80° C. for 10 minutes.

### EXAMPLE 5

Production of a Jelly Material:

A gelling agent solution was obtained by adding 0.3 part of carrageenan powder and 0.3 part of locust bean gum powder to 50 parts of water and dissolving by heating at about 80° C.

To the gelling agent solution just after heating under the above conditions, 41 parts of maltodextrin syrup which had been heated at about 60° C. for about 1 minute, 5 parts of the granular ε-polylysine B, 2.4 parts of a fruit flavor solution and 1 part of an acidifying agent were added to obtain a jelly base, followed by cooling to 55° C., and the jelly base was packed in a 60 cc capacity container and then cooled to obtain a jelly.

While the invention has been described in detail and with reference to specific embodiments thereof, it will be apparent to one skill in the art that various changes and modifications can be made therein without departing from the spirit and scope thereof. All references cited herein are incorporated in their entirety.

What is claimed is:

1. An oral agent for adsorbing phosphorus, which comprises a polylysine as an active ingredient.

2. The oral agent for adsorbing phosphorus according to claim 1, wherein the polylysine is crosslinked by a crosslinking agent.

3. The oral agent for adsorbing phosphorus according to claim 2, wherein the polylysine is crosslinked by a crosslinking agent and has a granular shape.

4. The oral agent for adsorbing phosphorus according to claim 2, wherein the polylysine is crosslinked by a crosslinking agent and has a plate shape.

5. The oral agent for adsorbing phosphorus according to claim 2, wherein the crosslinking agent is an epoxy compound.

6. The oral agent for adsorbing phosphorus according to claim 5, wherein the crosslinking agent is epichlorohydrin.

7. The oral agent for adsorbing phosphorus according to claim 5, wherein the crosslinking agent is ethylene glycol diglycidyl ether.

8. The oral agent for adsorbing phosphorus according to claim 1, wherein the polylysine is poly(α-lysine).

9. The oral agent for adsorbing phosphorus according to claim 1, wherein the polylysine is poly(ε-lysine).

10. A food which comprises the polylysine of claim 1.

11. The food according to claim 10, wherein the polylysine is crosslinked by a crosslinking agent.

12. The food according to claim 11, wherein the polylysine is crosslinked by a crosslinking agent and has a granular shape.

13. The food according to claim 11, wherein the polylysine is crosslinked by a crosslinking agent and has a plate shape.

14. The food according to claim 11, wherein the crosslinking agent is an epoxy compound.

15. The food according to claim 14, wherein the crosslinking agent is epichlorohydrin.

16. The food according to claim 14, wherein the crosslinking agent is ethylene glycol diglycidyl ether.

17. The food according to claim 10, wherein the polylysine is poly(α-lysine).

18. The food according to claim 10, wherein the polylysine is poly(ε-lysine).

19. A method for reducing a phosphorus concentration in blood, which comprises administering an effective amount of the oral agent for adsorbing phosphorus of claim 1 to human or an animal.

20. A method for excreting phosphorus, which comprises adsorbing phosphorus in the digestive tract by the oral agent for adsorbing phosphorus of claim 1 and allowing the phosphorus to be excreted together with the adsorbing agent.
